(19) United States
(12) Patent Application Publication
(10) Pub. No.: US 2026/0263255 A1
(43) Pub. Date: Sep. 10, 2026

(54) Non-Compressive Cervical Collars

(71) Applicant: The Regents of the University of California, Oakland, CA (US)

(72) Inventors: Yama Akbari, Irvine, CA (US); Christine King, Irvine, CA (US); Mohammed Osman, Valencia, CA (US); Ananth Bharadwaj, Valencia, CA (US); Alex Huang, Irvine, CA (US); Artin Rashidi, Irvine, CA (US); Ryan Johnson, Orange, CA (US)

(21) Appl. No.: 19/562,464

(22) Filed: Mar. 10, 2026

Related U.S. Application Data
(60) Provisional application No. 63/769,455, filed on Mar. 10, 2025.

(51) Int. Cl. A61F 5/055

(52) U.S. Cl. CPC A61F 5/055

(57) ABSTRACT

Non-compressive cervical collars are disclosed. In one embodiment, non-compressive cervical collar for stabilizing a user's neck while maintaining a clearance zone along sides of the user's neck is provided, the non-compressive cervical collar comprising: a chest section comprising a protrusion that is attached to at least one pillar; a first shoulder section and a second shoulder section; a jaw base that is attached to the at least one pillar, the jaw base comprising at least one jaw base attachment pad that attaches to at least one jaw strap, wherein the at least one jaw strap wraps around the user below a user's ears and above a user's shoulders without making contact with the sides of the user's neck; and a back section comprising a top back section, the top back section comprising a top back attachment pad that attaches to the at least one jaw strap.

### CROSS REFERENCE TO RELATED APPLICATION

The current application claims priority to U.S. Provisional Patent Application No. 63/769,455, filed on Mar. 10, 2025, the disclosure of which is incorporated herein by reference.

### FIELD OF THE INVENTION

The present invention generally relates to medical devices and more specifically to non-compressive cervical collars.

### BACKGROUND

A cervical collar is a medical device used to support and immobilize a person's neck. It may be utilized for patients with injuries such as whiplash, cervical fractures, and various other traumatic head or neck injuries that may comprise the cervical spinal cord. In particular, a cervical collar can be used to prevent further injury following an initial incident, including spinal cord injury. Additionally, it may be employed to treat chronic medical conditions and aid in post-surgical recovery.

### SUMMARY OF THE INVENTION

The various embodiments of the present non-compressive cervical collars contain several features, no single one of which is solely responsible for their desirable attributes. Without limiting the scope of the present embodiments, their more prominent features will now be discussed below. After considering this discussion, and particularly after reading the section entitled “Detailed Description,” one will understand how the features of the present embodiments provide the advantages described herein.

In a first aspect, a non-compressive cervical collar for stabilizing a user's neck while maintaining a clearance zone along sides of the user's neck is provided, the non-compressive cervical collar comprising: a chest section configured to receive a user's chest, the chest section comprising a protrusion that is attached to at least one pillar; a first shoulder section configured to receive a user's first shoulder and a second shoulder section configured to receive a user's second shoulder; a jaw base that is attached to the at least one pillar, the jaw base comprising at least one jaw base attachment pad that attaches to at least one jaw strap, wherein the at least one jaw strap wraps around the user below a user's ears and above a user's shoulders without making contact with the sides of the user's neck; and a back section comprising a top back section, the top back section comprising a top back attachment pad that attaches to the at least one jaw strap.

In an embodiment of the first aspect, the at least one pillar comprises a first pillar and a second pillar.

In another embodiment of the first aspect, the first and second pillars are separated by a distance creating an opening allowing access to a user's throat.

In another embodiment of the first aspect, the at least one jaw base attachment pad comprises a first jaw base attachment pad and a second jaw base attachment pad, and wherein the at least one jaw strap comprises a first jaw strap and a second jaw strap.

In another embodiment of the first aspect, the first jaw base attachment pad and the first jaw strap attach using a first jaw base hook-and-loop fastener system and the second jaw base attachment pad and the second jaw strap attach using a second jaw base hook-and-loop fastener system.

In another embodiment of the first aspect, the top back section comprises at least one top back slot.

In another embodiment of the first aspect, the non-compressive cervical collar further comprises a head strap that wraps around the user's head, wherein the at least one top back slot receives the head strap and the head strap folds and attaches onto itself.

In another embodiment of the first aspect, the head strap attaches onto itself using a hook-and-loop fastener system.

In another embodiment of the first aspect, the back section comprises a bottom back section, wherein the top back section receives a head of the user and the bottom back section receives a back of the user.

In another embodiment of the first aspect, the first shoulder section comprises a first front shoulder section and a first back shoulder section.

In another embodiment of the first aspect, the first front shoulder section and the first back shoulder section are connected.

In another embodiment of the first aspect, the first front and back shoulder sections are connected using a first shoulder strap.

In another embodiment of the first aspect, the second shoulder section comprises a second front shoulder section and a second back shoulder section.

In another embodiment of the first aspect, the second front shoulder section and the second back shoulder section are connected.

In another embodiment of the first aspect, the second front and back shoulder sections are connected using a second shoulder strap.

In another embodiment of the first aspect, the jaw base further comprises a chin dip for receiving a user's chin.

In another embodiment of the first aspect, the chin dip includes a cavity allowing a mouth of the user to open and close.

In another embodiment of the first aspect, the chest section, the first and second shoulder sections, the jaw base, and the back section include padding for user comfort.

In another embodiment of the first aspect, the chest section, first front and back shoulder sections, the second front and back shoulder sections, the jaw base, the first and second pillars, the top back section, the bottom back section are modular components.

In another embodiment of the first aspect, the chest section comprises a first and second chest section that are modular components and mirror images of each other, the bottom back section comprises a first and second bottom back section that are modular components and mirror images of each other, the top back section comprises a first and second top back section that are modular components and mirror images of each other.

### BRIEF DESCRIPTION OF THE DRAWINGS

The various embodiments of the present non-compressive cervical collars now will be discussed in detail with an emphasis on highlighting the advantageous features. These embodiments depict the novel and non-obvious non-compressive cervical collars shown in the accompanying drawings, which are for illustrative purposes only. These drawings include the following figures:

FIG. 1A is a diagram illustrating a front view of a non-compressive cervical collar on a user in accordance with an embodiment of the invention.

FIG. 1B is a diagram illustrating a side view of a non-compressive cervical collar on a user in accordance with an embodiment of the invention.

FIG. 1C is a diagram illustrating a back view of a non-compressive cervical collar on a user in accordance with an embodiment of the invention.

FIG. 2A is a diagram illustrating an exploded front view of a non-compressive cervical collar for modular construction in accordance with an embodiment of the invention.

FIG. 2B is a diagram illustrating an exploded back view of a non-compressive cervical collar for modular construction in accordance with an embodiment of the invention.

FIG. 2C is a diagram illustrating an exploded left-side view of a non-compressive cervical collar for modular construction in accordance with an embodiment of the invention.

FIG. 2D is a diagram illustrating an exploded right-side view of a non-compressive cervical collar for modular construction in accordance with an embodiment of the invention.

FIG. 3A is a computer-aided design (“CAD”) diagram illustrating a trimetric view of a non-compressive cervical collar in accordance with an embodiment of the invention.

FIG. 3B is a CAD diagram illustrating a front view of a non-compressive cervical collar in accordance with an embodiment of the invention.

FIG. 3C is a CAD diagram illustrating a back view of a non-compressive cervical collar in accordance with an embodiment of the invention.

FIGS. 4A-D are CAD diagrams illustrating an axonometric projection, a right-side view, a top view, and a back view, respectively, of a first chest section of a non-compressive cervical collar in accordance with an embodiment of the invention.

FIGS. 5A-D are CAD diagrams illustrating an axonometric projection, a right-side view, a top view, and a back view, respectively, of a second chest section of a non-compressive cervical collar in accordance with an embodiment of the invention.

FIGS. 6A-D are CAD diagrams illustrating an axonometric projection, a right-side view, a top view, and a front view, respectively, of back shoulder section (e.g., a first or second back shoulder section) of a non-compressive cervical collar in accordance with an embodiment of the invention.

FIGS. 7A-D are CAD diagrams illustrating an axonometric projection, a left-side view, a top view, and a front view, respectively, of a front shoulder section (e.g., a first or second front shoulder section) of a non-compressive cervical collar in accordance with an embodiment of the invention.

FIGS. 8A-D are CAD diagrams illustrating an axonometric projection, a right-side view, a top view, and a front view, respectively, of a bottom back section (e.g., a first or second bottom back section) of a non-compressive cervical collar in accordance with an embodiment of the invention.

FIGS. 9A-D are CAD diagrams illustrating an axonometric projection, a right-side view, a top view, and a back view, respectively, of a first top back section of a non-compressive cervical collar in accordance with an embodiment of the invention.

FIGS. 10A-D are CAD diagrams illustrating an axonometric projection, a right-side view, a top view, and a back view, respectively, of a second top back section of a non-compressive cervical collar in accordance with an embodiment of the invention.

FIGS. 11A-D are CAD diagrams illustrating an axonometric projection, a right-side view, a bottom view, and a front view, respectively, of a jaw base of a non-compressive cervical collar in accordance with an embodiment of the invention.

FIGS. 12A-D are CAD diagrams illustrating an axonometric projection, a right-side view, a top view, and a front view, respectively, of a first pillar of a non-compressive cervical collar in accordance with an embodiment of the invention.

FIGS. 13A-D are CAD diagrams illustrating an axonometric projection, a right-side view, a top view, and a front view, respectively, of a second pillar of a non-compressive cervical collar in accordance with an embodiment of the invention.

### DETAILED DESCRIPTION OF THE DRAWINGS

The various embodiments of the present non-compressive cervical collars contain several features, no single one of which is solely responsible for their desirable attributes. Without limiting the scope of the present embodiments, their more prominent features will now be discussed below. After considering this discussion, and particularly after reading this section entitled “Detailed Description of the Drawings,” one will understand how the features of the present embodiments provide the advantages described here.

One aspect of the present embodiments includes the realization that in using conventional cervical collars, other than the present embodiments, a user's jugular vein is compressed. The jugular vein's primary function is to carry blood from the head and neck to the heart. Jugular vein compression can occur when the jugular veins on either side of the neck are pinched or compressed at all, a common occurrence with conventional cervical collars. Due to the heavy neck contact with conventional cervical collars, such cervical collars impede blood flow, causing a backflow of blood back to the head. The blood volume in the skull may rise, leading to various negative conditions such as, but not limited to, an increased intracranial pressure (ICP). An increase in ICP may cause signs and symptoms ranging from headaches and brain fog to reduction of blood flow and brain herniation, which may lead to irreversible brain damage or death. For example, the unexpected increase in cerebral blood volume may compress brain tissue, escalating the potential for brain herniation, neurological damage, and even brain death. This process can be gradual or rapid. Further, patients with severe brain injury are often comatose and may be unable to express any discomfort, presenting a complex problem, especially when invasive ICP monitors are not in place. Many studies have observed that jugular vein compression is harmful to any patient, especially those who are already suffering from acute brain injury due to trauma, stroke, or other neurological conditions. A neck injury (e.g., a neck fracture) may include many deleterious risks for the patient but if jugular vein compression is combined with this affliction, the subsequent potential for brain injury can lead to severe suffering of the patient, increasing the likelihood of morbidity and mortality. This is the reality for patients who wear the prescribed conventional cervical collar in the setting of acute brain injury. Even in patients with cervical injuries in the absence of brain injury, the effects of jugular vein compression may also lead to altered cerebral hemodynamics and consequent neurological issues. For example, compression of the jugular veins could affect the brain's innate clearance mechanism of toxins through untoward modulation of the glymphatic system and/or the meningeal lymphatic system. Moreover, it is also possible that conventional cervical collars compressing the sides of the neck could compress on the carotid arteries. While arteries are more rigid than veins and less prone to compression, significant tightening or pressure by compressible cervical collars could reduce arterial flow to the brain, leading to ischemia. Furthermore, in the event that a patient has atherosclerotic plaques in the carotid artery region that is being compressed, the possibility exists of ischemia and/or embolization of plaque components (e.g. increased risk of plaque rupture from compression) leading to a stroke. As such, avoidance of any compression on either arteries or veins of the neck would reduce these risks.

The present embodiments solve these problems by immobilizing or stabilizing a user's neck without making contact with sides of the user's neck. For example, the present embodiments may include non-compressive cervical collars that maintain a clearance zone along the sides of the neck, as further described below. The present embodiments thus advantageously enable jugular venous blood flow and carotid artery blood flow while essentially immobilizing the user's neck.

Another aspect of the present embodiments includes the realization that conventional cervical collars may be bulky and not optimized for logistics such as, but not limited to, manufacture, boxing, shipping, storage, etc. For example, conventional cervical collars are often shaped to be supported by a user's body and/or shaped to create space to receive a user's body part. Such design features may provide added structural support for the neck but may result in cervical collars that are relatively large. The present embodiments solve this problem by providing a modular construction with various parts that are interconnecting and optimized for logistics considerations. The present embodiments thus advantageously enable optimized manufacturing, boxing, shipping, and storing. The present embodiments provide these advantages and enhancements, as described below.

Turning now to the drawings, non-compressive cervical collars for stabilization and/or immobilization (the terms “stabilization” and “immobilization” may be used interchangeable herein) of a user's neck that maintain a clearance zone along the sides of the neck preventing contact with the sides of the user's neck in accordance with embodiments of the invention are provided. As further described above, conventional cervical collars distribute forces by relying on the sides of the neck for immobilization. The present embodiments redirect these forces to prevent jugular vein or carotid artery compression. In several embodiments, non-compressive cervical collars may concentrate forces on the bony structures of the body. For example, forces for neck immobilization may be concentrated on a user's jaw, particularly for controlling rotational movements and thereby more efficiently preventing movement of the upper cervical vertebrae. Further, forces for neck immobilization may also be concentrated on the back of the head and chin to prevent flexion and extension movement, regulated by the cervical spine and its associated vertebrae. In addition, forces for neck immobilization may also be concentrated on a user's shoulders and temples to prevent lateral flexion, regulated by the cervical spine and its associated vertebrae. Finally, additional regions of the head and shoulder can be recruited to provide more stabilization as needed. For example, in some embodiments, we can incorporate recruitment of the mastoid bone, maxillary region, and other regions of the head. Having options to leverage other regions of the head and shoulder can provide flexibility in case some patients, such as trauma patients, have specific regions of the head or body that cannot tolerate much pressure. Such options may also prove useful in cases where multimodal brain monitoring is required, wherein probes need to be placed on certain regions (e.g. optical or electrical brain monitoring devices placed on the forehead). In such cases, the various embodiments described here can offer options while still maintaining immobilization of the neck.

In many embodiments, non-compressive cervical collars may include various components for front and back support. In some embodiments, the non-compressive cervical collars may include a jaw base that may be supported by one or more chin supports (may also be referred to as “pillars”). In some embodiments, the pillars may be connected to a chest section. In various embodiments, the non-compressive cervical collars may also include various straps for providing support. For example, non-compressive cervical collars may include a first strap (may also be referred to as the “jaw strap”) that may be connected to the jaw base, wherein the first strap wraps around the user below the user's ears and above the user's shoulders without making contact with the sides of the user's neck. Further, the non-compressive cervical collars may include a second strap (may also be referred to as the “head strap”) that wraps around the head. In some embodiments, the non-compressive cervical collars may also include two shoulder straps linked to the chest section. In several embodiments, the various straps may be equipped with an attachment mechanism that allows for reusable closure such as, but not limited to, a hook-and-loop fastener system (e.g., Velcro), snap fasteners, buttons, zippers, magnetic closures, adhesive strips, buckle systems, ties or laces, etc. In many embodiments, the attachment mechanism may ensure an adjustable and comfortable fit. Together, these components synergize to restrict neck flexion and extension, with the jaw base and jaw strap inhibiting neck rotation, and the head strap limiting lateral flexion. By maintaining a clearance zone along the sides of the neck preventing contact with the neck's sides, the present embodiments not only prevent jugular vein compression but also enhance stabilization and comfort, ultimately promoting quicker recovery without the risk of neurological damage. Non-compressive cervical collars in accordance with embodiments of the invention are further discussed below.

### Non-Compressive Cervical Collars

Non-compressive cervical collars may be considered as a redesign of the conventional cervical collar to enhance emergency health care by effectively eliminating the risk of jugular vein compression. In many embodiments, non-compressive cervical collars may maintain a clearance zone along the sides of the neck. For example, maintaining a clearance zone along the sides of the neck may include maintaining a clearance zone along the sides of the neck sufficient in space to accommodate wide anatomic variability in vascular structures and body habitus in individuals.

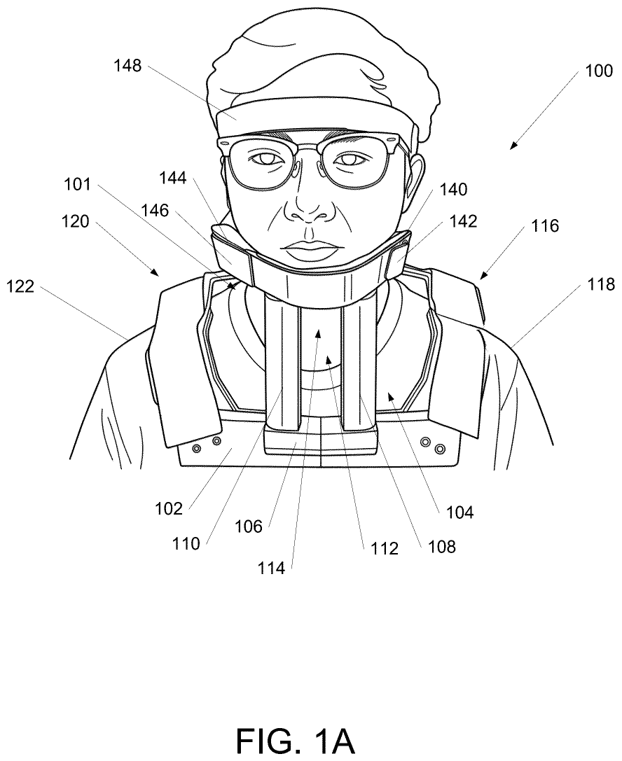

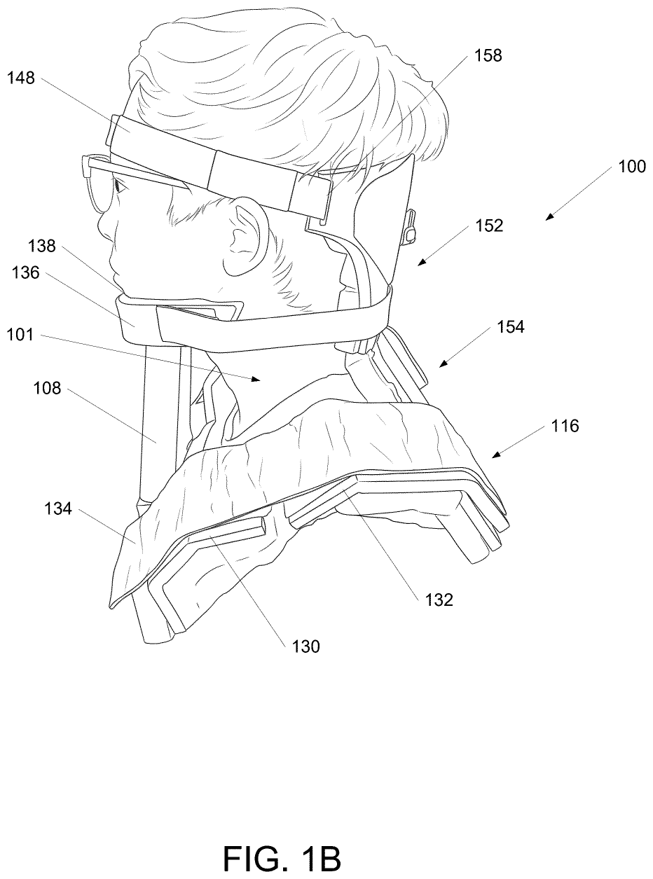

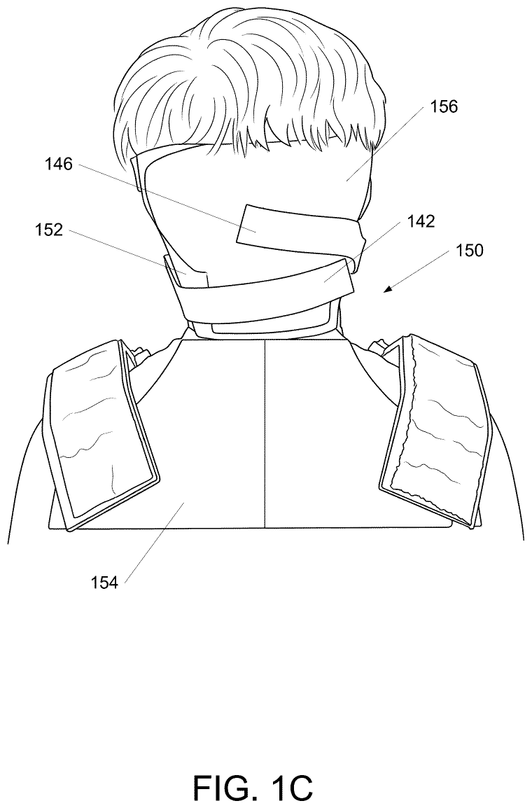

Diagrams illustrating a front, side, and back views of a non-compressive cervical collar on a user in accordance with an embodiment of the invention is shown in FIGS. 1A-C, respectively. A diagram illustrating a front view of a non-compressive cervical collar on a user in accordance with an embodiment of the invention is shown in FIG. 1A. The non-compressive cervical collar 100 may include a chest section 102 configured to receive a user's chest 104. In some embodiments, the chest section 102 may include a protrusion 106 that is attached to at least one pillar such as, but not limited to, a first pillar 108 and a second pillar 110. In some embodiments, the first and second pillars 108, 110 may be separated by a distance or gap 112 that allows access to the user's throat area 114. Access to the user's throat area 114 may be beneficial for various patients, including, but not limited to, patients that may require tracheostomy, treatment of lacerations in the throat area, etc.

In further reference to FIG. 1A, the non-compressive cervical collar 100 may include a first shoulder section 116 configured to receive a user's first shoulder 118 and a second shoulder section 120 configured to receive a user's second shoulder 122. In some embodiments, the first and second shoulder sections 116, 120 may be attached to the chest section 102, as further described below.

A diagram illustrating a side view of a non-compressive cervical collar on a user in accordance with an embodiment of the invention is shown in FIG. 1B. In some embodiments, the first shoulder section 116 may include a first front shoulder section 130 and a first back shoulder section 132. In some embodiments, the first front and back shoulder sections 130, 132 may be connected using a first shoulder strap 134. In some embodiments, first front and back shoulder sections 130, 132 and the first shoulder strap 134 may attach using a hook-and-loop fastener system (e.g., Velcro), snap fasteners, buttons, zippers, magnetic closures, adhesive strips, buckle systems, ties or laces, etc., where the attachment stabilizes the first front and back shoulder sections 130, 132 to a particular user's shoulder size. Likewise, the second shoulder section 120 may include a second front shoulder section and a second back shoulder section. In some embodiments, the second front and back shoulder sections may be connected using a second shoulder strap. In some embodiments, second front and back shoulder sections and the second shoulder strap may attach using a hook-and-loop fastener system (e.g., Velcro), snap fasteners, buttons, zippers, magnetic closures, adhesive strips, buckle systems, ties or laces, etc., where the attachment stabilizes the second front and back shoulder sections to a particular user's shoulder size. In some embodiments, the chest section 102, the first shoulder section 116, and the second shoulder section may include padding for user comfort, as further described below.

In reference to FIGS. 1A-B, the non-compressive cervical collar 100 may also include a jaw base 136 that is attached to at least one pillar (e.g., the first and second pillars, 108, 110). In some embodiments, the jaw base 136 may be configured to receive the user's jaw or portion of the user's jaw such as, but not limited to, the user's lower jaw. In some embodiments, the jaw base 136 may include a chin dip 138 configured to receive the user's chin. In some embodiments, the chin dip 138 may include space (may also be referred to as a “cavity”) that allows a user to open and/or close their mouth. For example, the chin dip may include a cavity that allows the user to open and/or close their mouth for receiving and processing foods.

In further reference to FIGS. 1A-B, the jaw base 136 may include a first jaw base attachment pad 140 that receives a first jaw strap 142 and a second jaw base attachment pad 144 that receives a second jaw strap 146. For example, the first jaw strap 142 may attach to the first jaw base attachment pad 140 and the top back attachment pad 156, as further described below. Similarly, the second jaw strap 146 may attach to the second jaw base attachment pad 144 and the top back attachment pad 156, as further described below. In various embodiments, the first and/or second jaw strap(s) 142, 146 may each wrap around one side of the user below the user's ears and above the user's shoulders without contacting the user's neck. In some embodiments, the first jaw base attachment pad 140 and the first jaw strap 142 may attach using a first jaw base attachment mechanism including, but not limited to, a first jaw base hook-and-loop fastener system (e.g., Velcro), snap fasteners, buttons, zippers, magnetic closures, adhesive strips, buckle systems, ties or laces, etc. In some embodiments, the second jaw base attachment pad 144 and the second jaw strap 146 may attach using a second jaw base attachment mechanism including, but not limited to, a second jaw base hook-and-loop fastener system (e.g., Velcro), snap fasteners, buttons, zippers, magnetic closures, adhesive strips, buckle systems, ties or laces, etc. In some embodiments, the jaw base 136 may include padding for user comfort, as further described below. In addition, the non-compressive cervical collar may include a head strap 148 for securing the user's head with a top back section 152 having a first top back slot 158 and/or a second top back slot, as further described below. As further described above, non-compressive cervical collars may maintain a clearance zone 101 along the sides of the neck, utilizing key contact points on the jaw, back of the head, and shoulders to provide neck stabilization. For example, maintaining a clearance zone 101 along the sides of the neck may include maintaining a clearance zone 101 along the sides of the neck sufficient in space to accommodate wide anatomic variability in vascular structures and body habitus in individuals.

A diagram illustrating a back view of a non-compressive cervical collar on a user in accordance with an embodiment of the invention is shown FIG. 1C. The non-compressive cervical collar 100 may include a back section 150 configured to receive a user's back, the back of the user's neck, and/or the back of the user's head. In some embodiments, the back section 150 may include a top back section 152 and a bottom back section 154. For example, the top and bottom back sections 152, 154 may contour to the back of the user's neck and the user's back, respectively. In many embodiments, the back section 150 may include a top back attachment pad 156 (e.g., a Velcro pad) that receives and attaches with the first jaw strap 142 and the second jaw strap 146 via a top back attachment mechanism. For example, the top back attachment mechanism may include, but is not limited to, a top back hook-and-loop fastener system (e.g., Velcro), snap fasteners, buttons, zippers, magnetic closures, adhesive strips, buckle systems, ties or laces, etc.

In reference to FIGS. 1B-C, the top back section 152 may include one or more back slots 158 (e.g., a first top back slot and/or a second back top slot) that receives the head strap 148 in securing the user's head. In embodiments with a first and second top back slot, the head strap 148 may wrap across the user's forehead, be inserted into the first and second top back slots, looped back towards the user's forehead, and secured to itself. In embodiments with only one top back slot, the head strap 148 may be fixed to the top back section 152 on a side of the user's head that is opposite of the position of the single top back slot. When used to secure the user's head, the head strap 148 may wrap across the user's forehead, be inserted into the top back slot (e.g., the top back slot 158), loop back towards the user's forehead, and secured to itself. In various embodiments, the head strap 148 may secure onto itself using a head strap attachment mechanism including, but not limited to, a hook-and-loop fastener system (e.g., Velcro), snap fasteners, buttons, zippers, magnetic closures, adhesive strips, buckle systems, ties or laces, etc. In some embodiments, the back section 150 may include padding for user comfort, as further described below.

Although specific non-compressive cervical collars are discussed above with respect to FIGS. 1A-C, any of a variety of non-compressive cervical collars including a variety of sections, straps, and attachment mechanisms, as appropriate to the requirements of a specific application can be utilized in accordance with embodiments of the invention. For example, one of ordinary skill would appreciate that various straps and the attachment mechanisms may be utilized with the present embodiments without deviating from the invention. For example, the present embodiments may utilize a single jaw strap instead of the first and second jaw straps. Further, the present embodiments may utilize a single jaw base attachment pad instead of the first and second jaw base attachment pad. Likewise, the present embodiments may utilize first and second head straps instead of the single head strap. Moreover, the present embodiments may utilize one top back slot (or no top back slots) in attaching the head strap. In addition, patients who need stabilizing and/or immobilization of the neck may also suffer from head trauma, including fractures in the skull. In such cases, the present embodiments may not need both or even one of the back slots. Furthermore, the present embodiments include various features that make it such that if some sections were changed (e.g., remove 1 back slot, or 1 pillar in the front, or other parts) the present embodiments may provide stabilization still without necessarily pushing on a skull fracture or jaw fracture (mandible or maxilla). The present embodiments contemplate changes that allow for applicability in trauma patients who may have various injuries such as, but not limited to, multiple fractures. In many embodiments, the present embodiments may be practiced and implemented without one or more of the various straps to achieve cervical spine immobilization.

Further, instead of a hoop-and-loop fastener system (e.g., Velcro) as illustrated, any reusable closure such as, but not limited to, snap fasteners, buttons, zippers, magnetic closures, adhesive strips, buckle systems, ties or laces, etc. may be utilized in accordance with embodiments of the invention. Modular construction of non-compressive cervical collars in accordance with embodiments of the invention are discussed further below.

### Modular Design and Construction of Non-Compressive Cervical Collars

Modular construction of non-compressive cervical collars may allow for optimization of costs and logistics. For example, the present embodiments include consideration of parts that are advantageously interconnecting enabling optimized manufacturing, boxing, shipping, and storing. In addition, the module design of non-compressive cervical collars may provide for micro-adjustments between each use and/or between various users which may be an important feature for repeat use in the real world. In addition, the various attachment mechanisms of the non-compressive cervical collars may readily attach and detach allowing for micro-adjustments between each use and/or between use by various users.

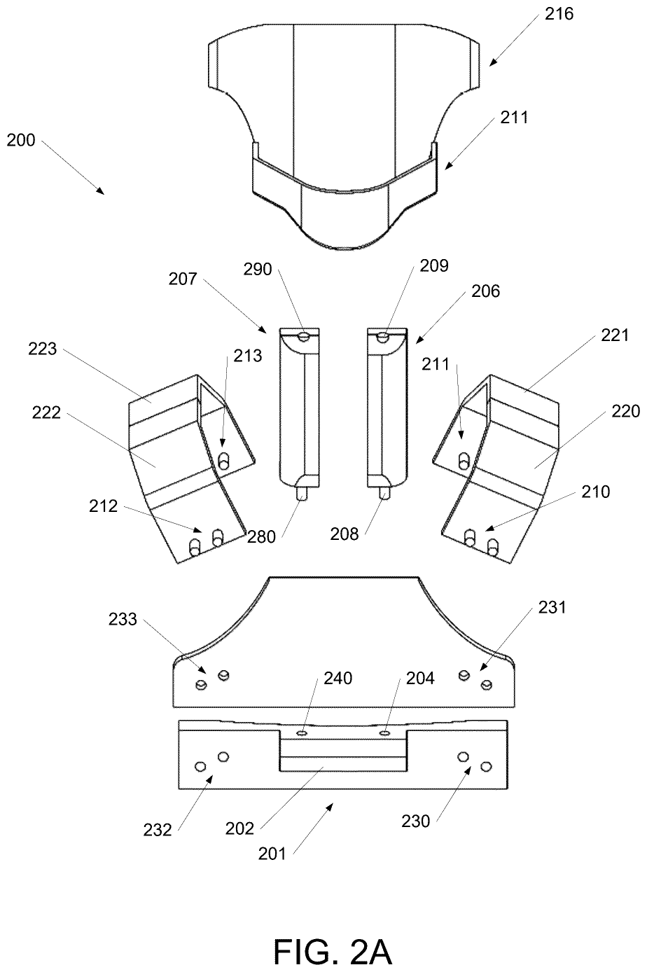

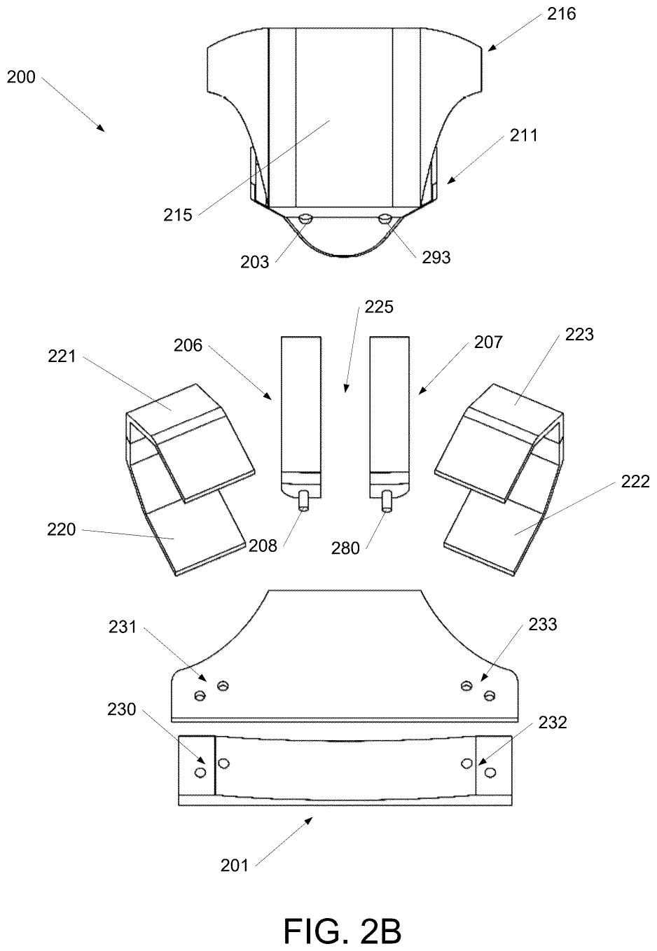

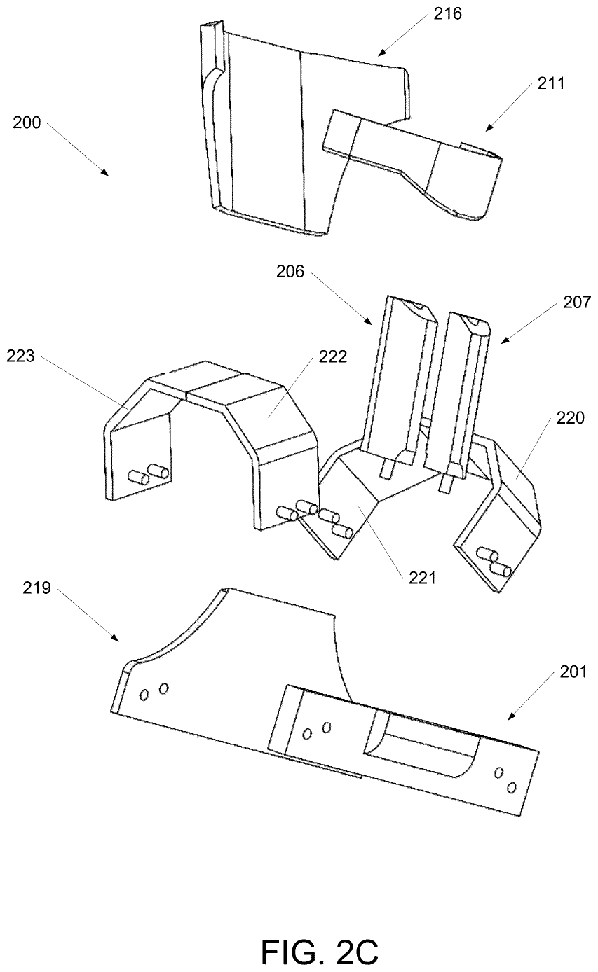

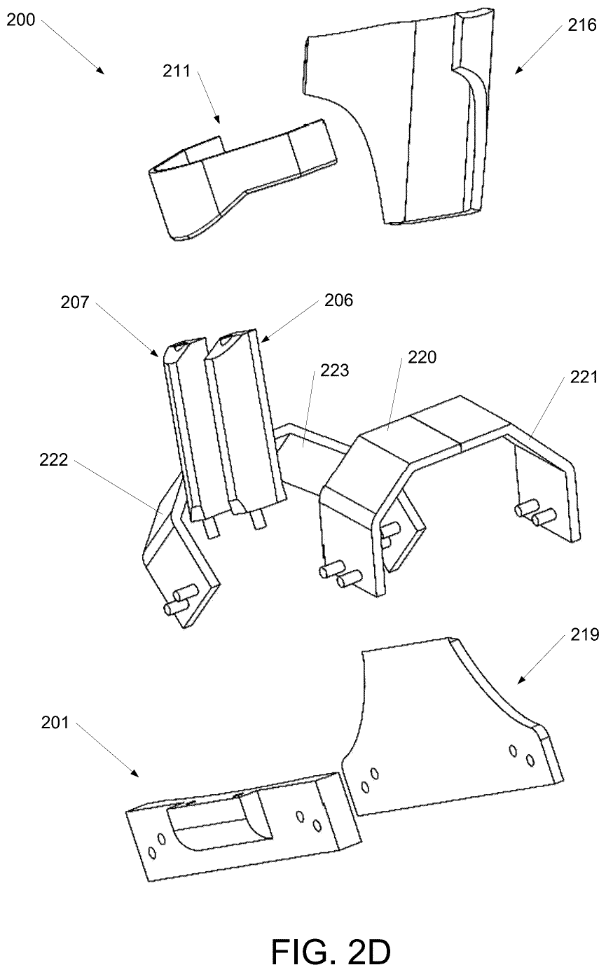

Diagrams illustrating an exploded front, back, left-side, and right-side views of a non-compressive cervical collar for modular construction in accordance with an embodiment of the invention are shown in FIGS. 2A-D, respectively. In reference to FIGS. 2C-D, the non-compressive cervical collar 200 may include a chest section 201 that may include a protrusion 202. In some embodiments, the chest section 201 and the protrusion 202 may be a singular piece. In some embodiments, the chest section 201 and the protrusion 202 may be separate pieces. In some embodiments, the chest section 201 and the protrusion may be symmetrical about a vertical center line. In some embodiments, the chest section 201 and the protrusion 202 may be split in half along the vertical center line as two separate pieces, as further described below.

In reference to FIGS. 2C-D, the non-compressive cervical collar 200 may also include a first pillar 206 and a second pillar 207. In some embodiments, the first and second pillars 206, 207 may attach to the protrusion 202, as further described below. In several embodiments, the non-compressive cervical collar 200 may also include a jaw base 211 that is attached to the first and second pillars 206, 207. In addition, the non-compressive cervical collar 200 may include a first shoulder that may include a first front shoulder 220 and a first back shoulder 221. Further, the non-compressive cervical collar 200 may include a second shoulder that may include a second front shoulder 222 and a second back shoulder 223. The first front shoulder 220 and the second front shoulder 222 may attach to the chest section 201, as further described below. The first back shoulder 221 and the second back shoulder 223 may attach to a bottom back section 219, as further described below. In some embodiments, the back section may include a top back section 216 and the bottom back section 219 (see FIG. 2B), as further described below. The top back section 216 may include a top back attachment pad 215 (e.g., a Velcro hook portion of a Velcro system), as further described herein. The straps (e.g., jaw strap, head strap, first and second shoulder straps) have been omitted from FIGS. 2A-D for clarity purposes.

In further reference to FIGS. 2C-D, the protrusion 202 may be adapted to extend outwards of the clavicle of a patient (may also be referred to as the “user”). In some embodiments, the chest section 201 may also include a padding such as, but not limited to, padding based on synthetic polymers (e.g., a polyurethane, etc.), natural fibers (e.g., coconut coir, natural tree latex, cotton, wool, etc.), or any mix of materials. For example, the padding (e.g., padded polyurethane foam) may be attached to chest section 201 and configured to rest against the body of the patient, namely against the patient's chest. Further, the chest section 201 and the extension 202 may include a continuous padding of polyurethane foam along the entire portion of the chest section 201, including the first and second front shoulder sections 220, 222, where the chest section 201 contacts the upper pecs (pectoralis major) of the patient and first and second front shoulder sections 220, 222 contact the upper trapezius of the patient. Otherwise, the chest section 201 and first and second front shoulder sections 220, 222, being made of a rigid material (e.g., rigid plastic material, 3D printer filament, etc.), could cause discomfort to the patient and might cause decubitus as unpadded portions of the chest section 201 and first and second front shoulder sections 220, 222 would rest against the body of the patient for long periods of time. In some embodiments, some or all parts of the chest section 201 and the first and second front shoulder sections 220, 222 may have padding (e.g., the polyurethane foam padding). In some embodiments, padding foam may be attached to the chest section 201 by releasable or non-releasable attachment mechanism such as, but not limited to, hook-and-loop fasteners, adhesives, rivets or other suitable attachment mechanisms.

As further described above, non-compressive cervical collars may often be used for trauma patients who have injuries other than those for which a cervical collar is used. For example, some patients may require a tracheostomy which might prevent use of a cervical collar which covers the throat. Accordingly, and as illustrated in FIGS. 2A-B, the first and second pillars 206, 207 may be positioned apart creating an opening 225 which allows for access to the throat of the patient. Typically, foam padding is not necessary since the first and second pillars 206, 207 do not come in contact with the patient.

In further reference to FIGS. 2A-B, the first front shoulder section 220 may include a first front shoulder peg 210. In some embodiments, the first front shoulder peg 210 may be a plurality of pegs (e.g., a pair of pegs). The first front shoulder peg(s) 210 may attach to corresponding hole(s) 230 in the chest section 201. For example, the first front shoulder peg(s) 210 may be constructed (e.g., 3D printed) to slip into the hole(s) 230 in the chest section 201 through friction. Similarly, the second front shoulder section 222 may include a second front shoulder peg 212. In some embodiments, the second front shoulder peg 212 may be a plurality of pegs (e.g., a pair of pegs). The second front shoulder peg(s) 212 may attach to corresponding hole(s) 232 in the chest section 201. For example, the second front shoulder peg(s) 212 may be constructed (e.g., 3D printed) to slip into the hole(s) 232 in the chest section 201 through friction. In some embodiments, the first and second front shoulder pegs 210, 212 may be positioned to project away from the patient's body when the non-compressive cervical collar 200 is in use. In other embodiments, first and second front shoulder pegs 210, 212 may be positioned to project towards the patient's body when the non-compressive cervical collar 200 is in use.

In further reference to FIGS. 2A-B, the first back shoulder section 221 may include a first back shoulder peg 211. In some embodiments, the first back shoulder peg 211 may be a plurality of pegs (e.g., a pair of pegs). The first back shoulder peg(s) 211 may attach to corresponding hole(s) 231 in the bottom back section 219. For example, the first back shoulder peg(s) 211 may be constructed (e.g., 3D printed) to slip into the hole(s) 231 in the bottom back section 219 through friction. Similarly, the second back shoulder section 223 may include a second back shoulder peg 213. In some embodiments, the second back shoulder peg 213 may be a plurality of pegs (e.g., a pair of pegs). The second back shoulder peg(s) 213 may attach to corresponding hole(s) 233 in the bottom back section 219. For example, the second back shoulder peg(s) 213 may be constructed (e.g., 3D printed) to slip into the hole(s) 233 in the bottom back section 219 through friction. In some embodiments, the first and second back shoulder pegs 211, 213 may be positioned to project towards the patient's body when the non-compressive cervical collar 200 is in use. In other embodiments, first and second back shoulder pegs 211, 213 may be positioned to project away from the patient's body when the non-compressive cervical collar 200 is in use.

In some embodiments, one or more of the holes 230-233 may be designed at various angles (e.g., a 45° angle) to achieve a friction anchoring with pegs 210-213, respectively. In some embodiments, additional adhesives such as, but not limited to, cyanoacrylate glue may be used to secure the first and second front shoulder sections 220, 222 with the chest section 201 and/or the first and second back shoulder sections 221, 223 with the bottom back section 219. In some embodiments, the padding (e.g., polyurethane foam padding) may be secured on the first and second front shoulder sections 220, 222 (excluding the first and second front shoulder pegs 210, 212) by releasable or non-releasable attachment mechanism such as, but not limited to, hook-and-loop fasteners, adhesives, rivets, or other suitable attachment mechanisms. Likewise, in some embodiments, the padding (e.g., polyurethane foam padding) may be secured on the first and second back shoulder sections 221, 223 (excluding the first and second back shoulder pegs 211, 213) by releasable or non-releasable attachment mechanism such as, but not limited to, hook-and-loop fasteners, adhesives, rivets, or other suitable attachment mechanisms.

In further reference to FIGS. 2A-B, the first and second front shoulder sections 220, 222 may connect to the first and second back shoulder sections 221, 223, respectively using first and second shoulder straps (not pictured in FIGS. 2A-D), respectively. For example, the first and second front shoulder sections 220, 222 and the first and second back shoulder sections 221, 223 may be coated with Velcro hooks. In some embodiments, the first and second shoulder straps may include Velcro loops (e.g., each may be a 6-inch strap of Velcro loops). In operation, the Velcro loops of the first and second shoulder straps may each attach to the Velcro hooks of the first and second front shoulder sections 220, 222, respectively. Then the Velcro loops of the first and second shoulder straps may be used to connect the first and second front shoulder sections 220, 222 to the first and second back shoulder sections 221, 223, respectively, by attaching and connecting to the first and second back shoulder sections 221, 223, respectively. In some embodiments, the padding (e.g., polyurethane foam padding) may be secured on the first and second front shoulder sections 220, 222 and the first and second back shoulder sections 221, 222 (excluding their pegs) by releasable or non-releasable attachment mechanisms including, but not limited to, hook-and-loop fasteners, adhesives, rivets, or other suitable attachment mechanisms.

In further reference to FIGS. 2A-B, the non-compressive cervical collar 200 may include a jaw base 211 having one or more hole(s) 203, 293 (see FIG. 2B). In some embodiments, the first pillar 206 may include one or more top peg(s) 209 and the second pillar 207 may include one or more top peg(s) 290. In various embodiments, the top pegs 209, 290 may attach to the one or more hole(s) 203, 293 located on the jaw base 211. In some embodiments, the top pegs 209, 290 may be configured to slip in the holes 203, 293 in jaw base 211 through friction. In some embodiments, additional adhesives such as, but not limited to, cyanoacrylate glue may be used to secure the jaw base 211 to the first and second pillars 206, 207. In some embodiments, the jaw base 211 may include a continuous foam padding along a chin dip, ridge, and extensions of the ridge. Otherwise, the jaw base 211, being made of a rigid material (e.g., plastic rigid material, 3D printer filament, etc.), could cause discomfort to the patient and might cause decubitus as the jaw and the side of the head of the patient would rest against unpadded portions of the jaw support for long periods of time.

In further reference to FIGS. 2A-B, the chest section 201 may include a protrusion 202 having one or more hole(s) 240, 204. In some embodiments, the first pillar 206 may include one or more bottom peg(s) 208 and the second pillar 207 may include one or more bottom peg(s) 280. In various embodiments, the bottom pegs 208, 280 may attach to the one or more hole(s) 204, 240 located on the protrusion 202. In some embodiments, the bottom pegs 208, 280 may be configured to slip in the pair of holes 204, 240 in protrusion 202 through friction. In some embodiments, additional adhesives such as, but not limited to, cyanoacrylate glue may be used to secure the protrusion 202 with the first and second pillars 206, 207. The protrusion 202 may be made of a rigid material (e.g., plastic rigid material, 3D printer filament, etc.)

In further reference to FIGS. 2A-D, the non-compressive cervical collar 200 may include a back section that may include a top back section 216 and bottom back section 219. As further described above, the bottom back section 219 may include one or more holes 231, 233 (e.g., pair of holes) designed at various angles (e.g., a 45° angle) for friction anchoring. As also further described above, the bottom back section 219 may be directly connected to the first and second back shoulder sections 221, 223 via their pegs 211, 213 and holes 231, 233, respectively, by various attachment mechanisms such as, but not limited to, a hook-and-loop fastener system (e.g., Velcro), snap fasteners, buttons, zippers, magnetic closures, adhesive strips, buckle systems, ties or laces, friction, and/or adhesive (e.g., cyanoacrylate glue), etc. In some embodiments, the padding (e.g., polyurethane foam padding) may be secured on the top back second 216 and/or the bottom back section 219 by releasable or non-releasable attachment mechanisms including, but not limited to, hook-and-loop fasteners, adhesives, rivets, or other suitable attachment mechanisms. In some embodiments, the top back second 216 and the bottom back section 219 may be directly connected using various attachment mechanisms such as, but not limited to, a hook-and-loop fastener system (e.g., Velcro), snap fasteners, buttons, zippers, magnetic closures, adhesive strips, buckle systems, ties or laces, friction, and/or an adhesive (e.g., cyanoacrylate glue). In some embodiments, the top back section 216 may include a top back attachment pad 215 having hooks (e.g., Velcro hook pad). In some embodiments, the top back attachment pad 215 may receive a first and/or second jaw strap having Velcro loops for securing and/or adjusting of the jaw strap(s), as further described above. For example, the first and second jaw straps may be two straps (e.g., two 3-inch straps) of Velcro loop that connect directly to the extension of the ridge in the jaw base 211 (e.g., via the first and second jaw base attachment pads, as further described above).

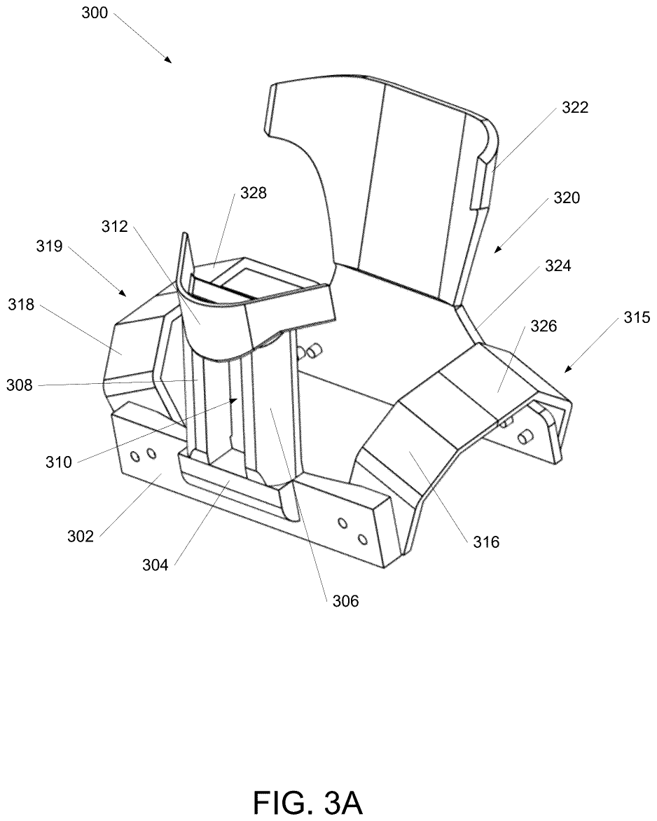

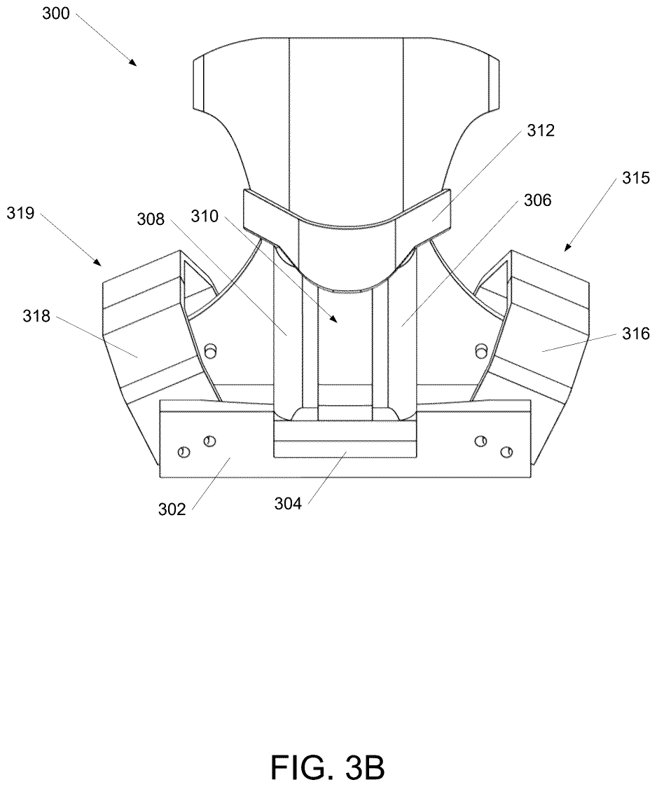

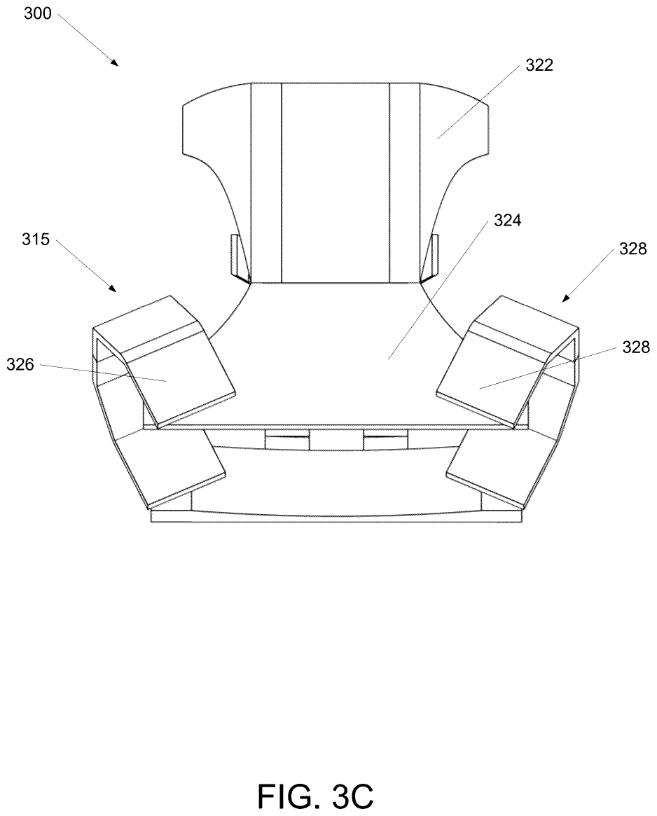

CAD diagrams illustrating a trimetric, front, and back views of a non-compressive cervical collar in accordance with an embodiment of the invention are shown in FIGS. 3A-C. The non-compressive cervical collar 300 is presented after assembly utilizing a modular construction, as further described above. The non-compressive cervical collar 300 includes a chest section 302 having a protrusion 304 that is attached to a first pillar 306 and a second pillar 308. Further, the first and second pillars 306, 308 are spaced to create an opening 310 to allow access to the user's throat. In addition, the first and second pillars 306, 308 may be attached, at the opposite end of the attachment with the protrusion 304, to a jaw base 312, as further described above. Moreover, the non-compressive cervical collar 300 may include a first shoulder section 315 having a first front shoulder section 316 and a first back shoulder section 326 that may be attached using a first shoulder strap 317, as further described above. Likewise, the non-compressive cervical collar 300 may include a second shoulder section 317 having a second front shoulder section 318 and a second back shoulder section 328 that may be attached using a second shoulder strap 321, as further described above.

In many embodiments, the chest section 302 may be attached to the first front shoulder section 316 at one end and to a second front shoulder section 318 at the other end. In some embodiments, the first and second front shoulder sections 316, 318 may include pegs that protrude away from the user's body and attach to the chest section 302 on the side of the chest section 302 that is closer to the user's body. However, in other embodiments, the first and second front shoulder sections 316, 318 may include pegs that protrude towards the user's body and attach to the chest section 302 on the side of the chest section 302 that is further away from the user's body.

In further reference to FIGS. 3A-C, the non-compressive cervical collar 300 may include a back section 320. In some embodiments, the back section 320 may include a top back section 322 and a bottom back section 324 that are contoured to the back of the user's head, the back of the user's neck, and/or the user's back. The bottom back section 324 may be attached to a first back shoulder section 326 at one end and to a second back shoulder section 328 at the other end. In some embodiments, the first and second back shoulder sections 326, 328 may include pegs that protrude towards the user's body and attached to the bottom back section 324 on the side of the bottom back section 324 that is further away from the user's body. However, in other embodiments, the first and second back shoulder sections 326, 328 may include pegs that protrude away from the user's body and attached to the bottom back section 324 on the side of the bottom back section 324 that is closer to the user's body.

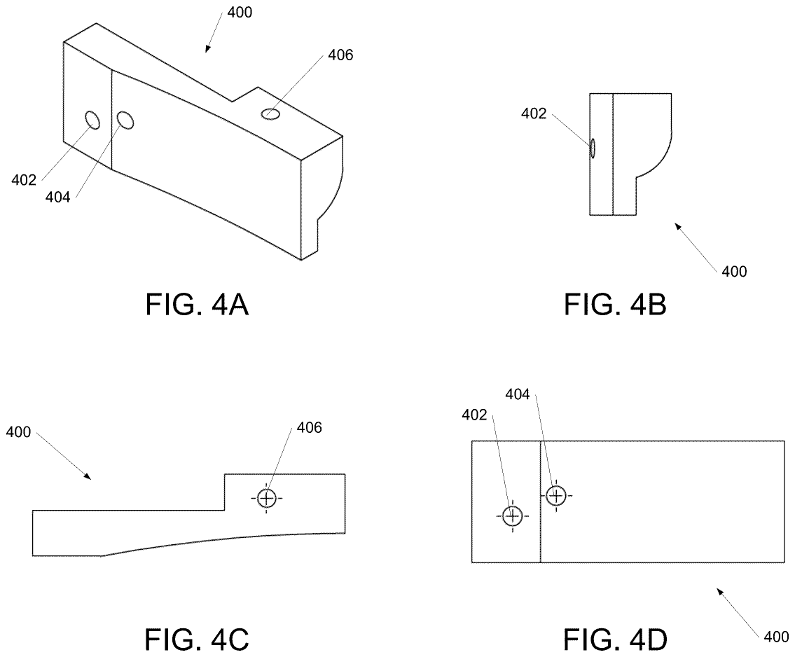

In many embodiments, the various sections and/or parts may be further divided into smaller parts in modular construction of non-compressive cervical collars. In particular, sections and/or parts may be designed for optimization with manufacturing processes including, but not limited to, 3D printing, injection molding, casting, forging, etc. CAD diagrams illustrating various sections and/or parts of a non-compressive cervical collar in accordance with an embodiment of the invention are illustrated in FIGS. 4A-13D.

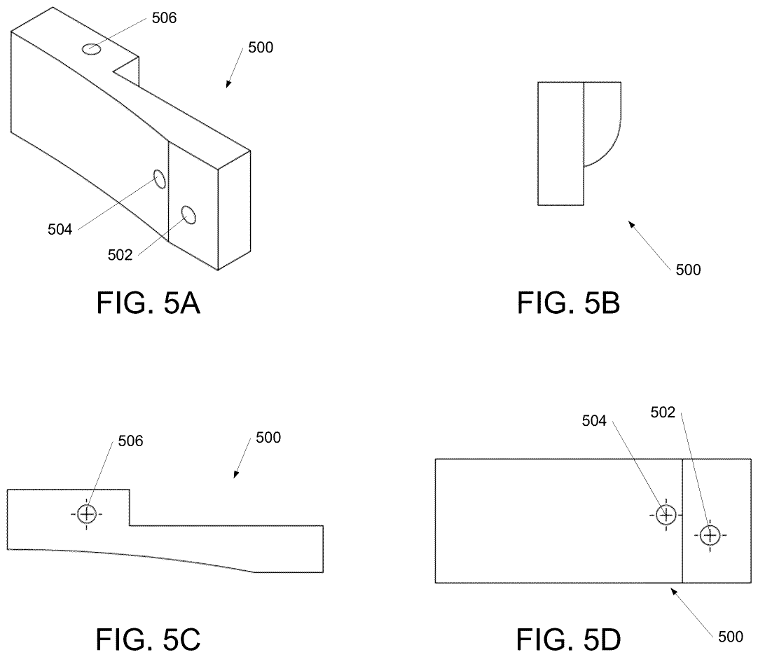

As further described above, a chest section may include a protrusion where the chest section and the protrusion may be symmetrical about a vertical center line. In some embodiments, a chest section may be split along the vertical center line resulting in the first chest section and the second chest section. CAD diagrams illustrating an axonometric projection, a right-side view, a top view, and a back view, respectively, of a first chest section of a non-compressive cervical collar in accordance with an embodiment of the invention are shown in FIGS. 4A-D. CAD diagrams illustrating an axonometric projection, a right-side view, a top view, and a back view, respectively, of a second chest section of a non-compressive cervical collar in accordance with an embodiment of the invention are shown in FIGS. 5A-D. In reference to FIGS. 4A-D, the first chest section 400 may include one or more holes 402, 404 for receiving peg(s) from a first front shoulder section, as further described above. Further, the first chest section 400 may also include one or more holes 406 for receiving one or more bottom peg(s) of a first pillar, as further described above. In reference to FIGS. 5A-D, the second chest section 500 may include one or more holes 502, 504 for receiving peg(s) from a second front shoulder section, as further described above. Further, the second chest section 500 may also include one or more holes 506 for receiving one or more bottom peg(s) of a second pillar, as further described above.

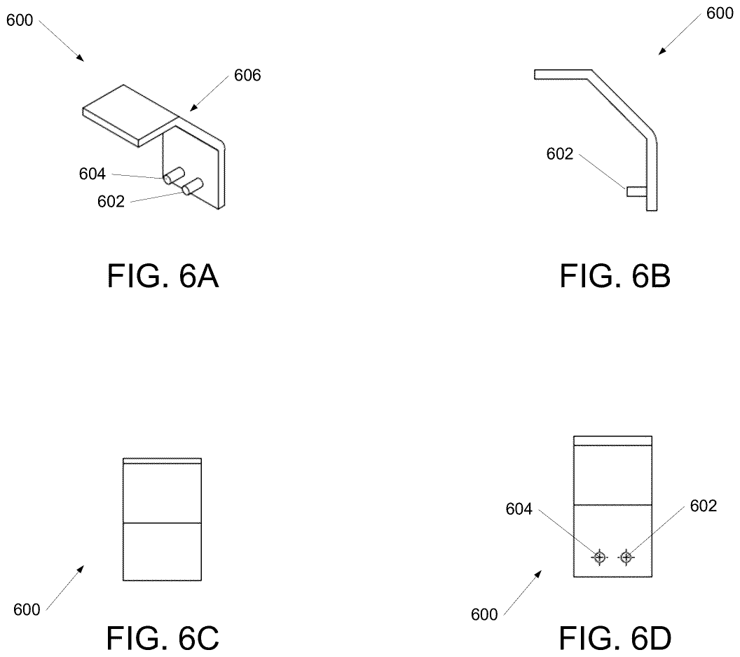

CAD diagrams illustrating an axonometric projection, a right-side view, a top view, and a front view, respectively, of back shoulder section (e.g., a first or second back shoulder section) of a non-compressive cervical collar in accordance with an embodiment of the invention are shown in FIGS. 6A-D. In many embodiments, the back shoulder section 600 may be utilized as the first back shoulder section and the second back shoulder section. For example, in some embodiments, the first and second back shoulder sections may be identical or mirror images of each other. In reference to FIGS. 6A-C, the first or second back shoulder section 600 may include one or more peg(s) 602, 604 that may be configured to attach to one or more holes of a bottom back section, as further described above. In some embodiments, the first or second back shoulder section 600 may include one or more bends 606 to contour to a back portion of the user's shoulder.

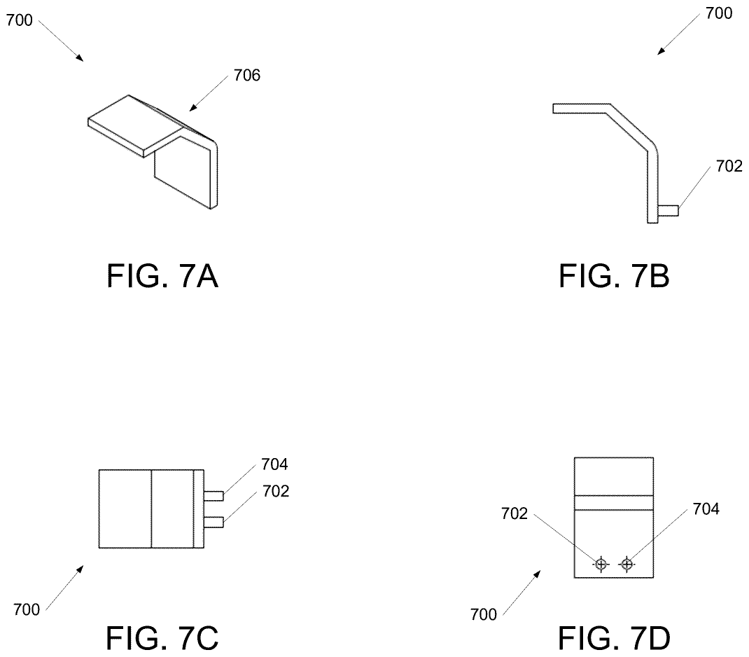

CAD diagrams illustrating an axonometric projection, a left-side view, a top view, and a front view, respectively, of a front shoulder section (e.g., a first or second front shoulder section) of a non-compressive cervical collar in accordance with an embodiment of the invention are shown in FIGS. 7A-D. In various embodiments, the front shoulder section 700 may be utilized as the first front shoulder section and the second front shoulder section. For example, in some embodiments, the first and second front shoulder sections may be identical or mirror images of each other. In reference to FIGS. 7A-D, the first or second front shoulder section 700 may include one or more peg(s) 702, 704 that may be configured to attach to one or more holes of a chest section, as further described above. In some embodiments, the first or second front shoulder section may include one or more bends 706 to contour to a front portion of the user's shoulder.

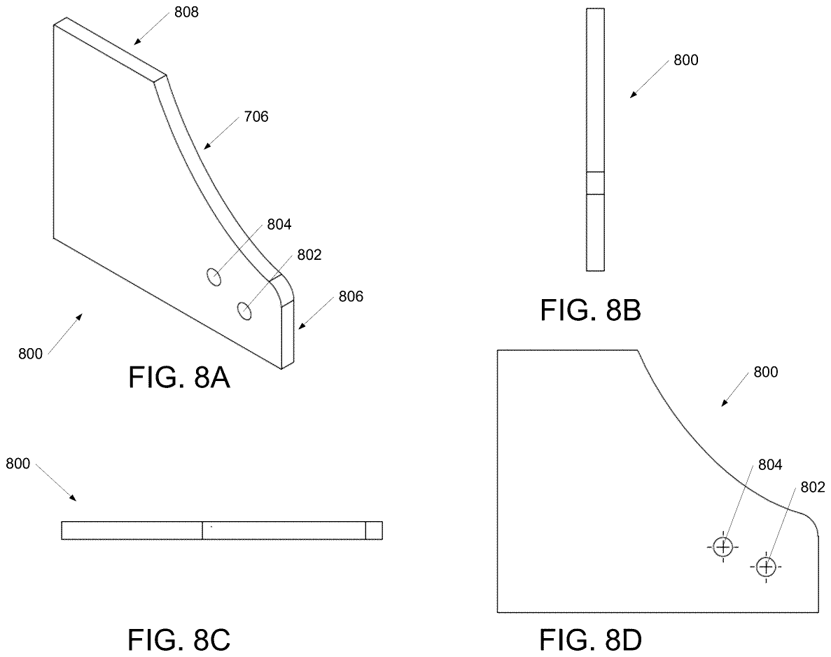

CAD diagrams illustrating an axonometric projection, a right-side view, a top view, and a front view, respectively, of a bottom back section (e.g., a first or second bottom back section) of a non-compressive cervical collar in accordance with an embodiment of the invention are shown in FIGS. 8A-D. In various embodiments, the bottom back section 800 may be utilized as the first bottom back section and the second bottom back section. For example, in some embodiments, the first and second back sections may be identical or mirror images of each other. In reference to FIGS. 8A-D, the first or second bottom back section 800 may include one or more holes 802, 804 for receiving peg(s) from the first or second back shoulder section, respectively, as further described above. In some embodiments, the first or second bottom back section 800 may include a lower portion 806 configured to receive the first or second back shoulder sections, respectively. In some embodiments, the first or second bottom back section 800 may also include an apex portion 808 configured to attached to a top back section, as further described above.

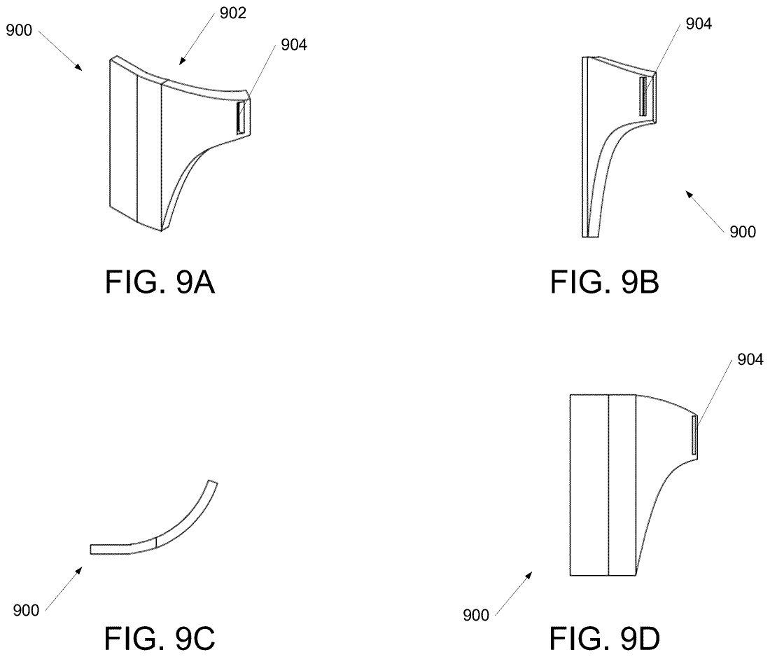

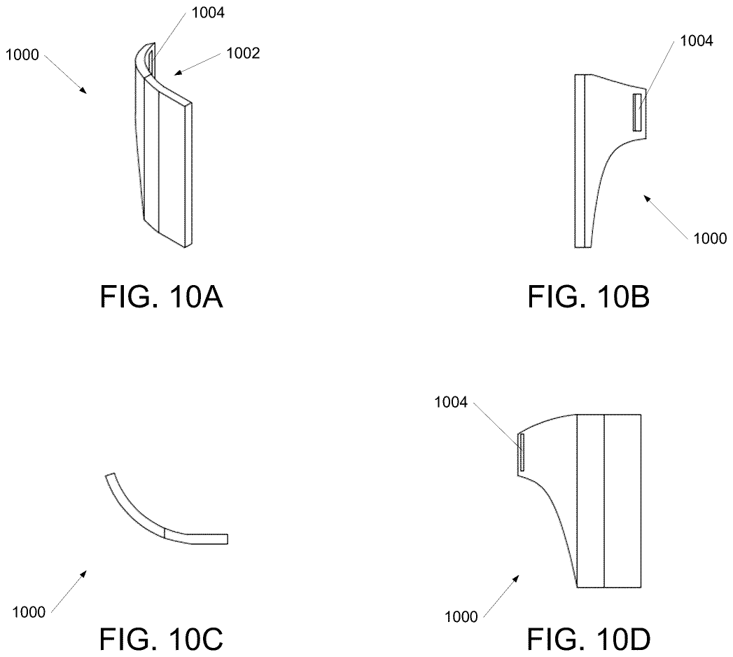

CAD diagrams illustrating an axonometric projection, a right-side view, a top view, and a back view, respectively, of a first top back section of a non-compressive cervical collar in accordance with an embodiment of the invention are shown in FIGS. 9A-D. CAD diagrams illustrating an axonometric projection, a right-side view, a top view, and a back view, respectively, of a second top back section of a non-compressive cervical collar in accordance with an embodiment of the invention are shown in FIGS. 10A-D. In some embodiments, the first and second top back sections 900, 1000 may be mirror images of each other. In some embodiments, the first and second top back sections 900, 1000 may include a one or more bends 902, 1002 to contour and/or support a back portion of a user's head. In some embodiments, the first and/or second top back sections 900, 1000 may include one or more slots 904, 1004 (may also be referred to as the “first top back slot” and the “second top back slot,” respectively) for receiving a head strap, as further described above. In some embodiments, the first and second top back sections 900, 1000 may attach to the first and second bottom back sections, as further described above.

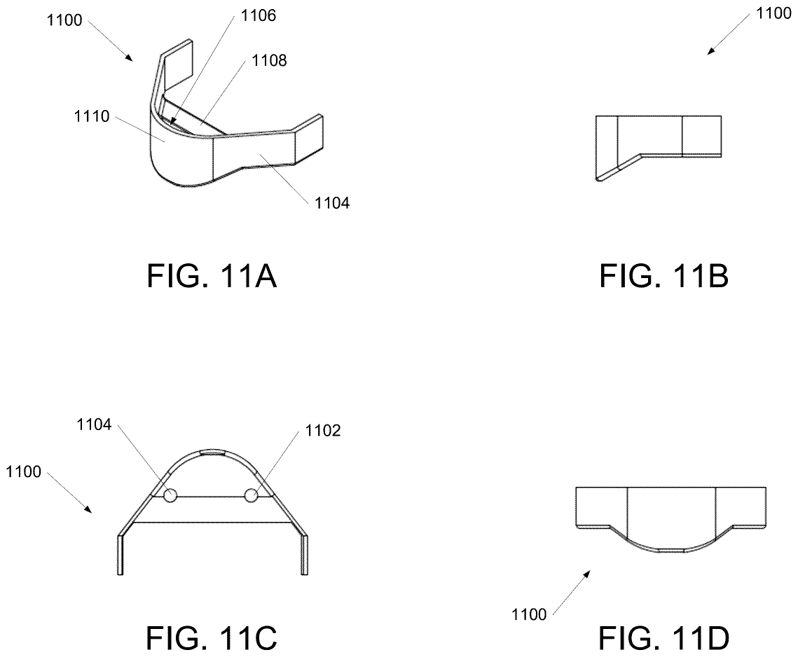

CAD diagrams illustrating an axonometric projection, a right-side view, a bottom view, and a front view, respectively, of a jaw base of a non-compressive cervical collar in accordance with an embodiment of the invention are shown in FIGS. 11A-D. The jaw base 1100 may also include one or more holes 1102, 1004 for receiving one or more top peg(s) of a first pillar and a second pillar, as further described above. In some embodiments, the jaw base 1100 may include a chin dip 1110 having a cavity 1106, ridge 1108, and/or extensions 1110 of the ridge, as further described above.

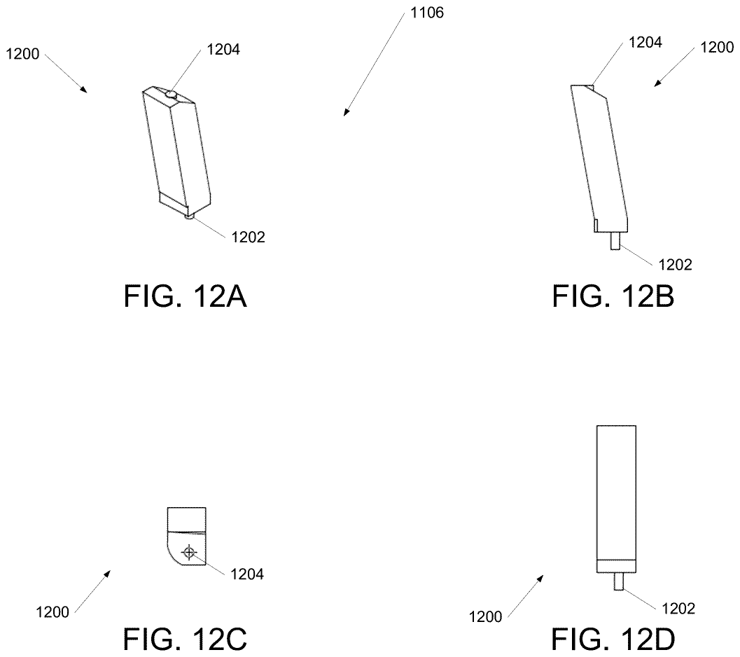

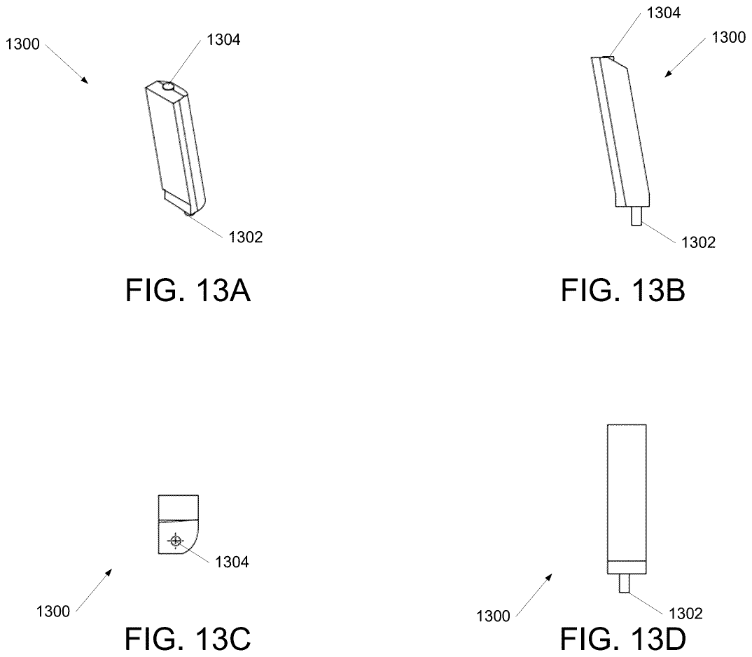

CAD diagrams illustrating an axonometric projection, a right-side view, a top view, and a front view, respectively, of a first pillar of a non-compressive cervical collar in accordance with an embodiment of the invention are shown in FIGS. 12A-D. CAD diagrams illustrating an axonometric projection, a right-side view, a top view, and a front view, respectively, of a second pillar of a non-compressive cervical collar in accordance with an embodiment of the invention are shown in FIGS. 13A-D. In some embodiments, the first and second pillars 1200, 1300 may include one or more bottom pegs 1202, 1302, respectively, configured to attached to one or more holes of a chest section, as further described above. In some embodiments, the first and second pillars 1200, 1300 may also include one or more top pegs 1204, 1304, respectively, configured to attached to one or more holes of a jaw base, as further described above. In some embodiments, the one or more top pegs 1204, 1304 may be shorter relative to the one or more bottom pegs 1202, 1302. Further, the first and/or second pillars 1200, 1300 may be tilted at an angle towards the user's chin when attached to the chest section.

Although specific non-compressive cervical collars for modular construction are discussed above with respect to FIGS. 2A-4J, any of a variety of non-compressive cervical collars for modular construction including differing configurations of the sections and/or components as appropriate to the requirements of a specific application can be utilized in accordance with embodiments of the invention. For example, in some embodiments, the first front and back shoulder sections may be a single first shoulder section. Similarly, the second front and back shoulder sections may be a single second shoulder section. Further, in some embodiments, the first and second shoulder sections may be combined with the chest section and/or the back section. Moreover, in some embodiments, the top and bottom back sections may be a single back section. Furthermore, the first and/or second pillars may be combined with the chest section and/or the jaw base.

Each of these non-limiting examples can stand on its own or can be combined in various permutations or combinations with one or more of the other examples. The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” In this document, the term “set” or “a set of” a particular item is used to refer to one or more than one of the particular item.

Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. In the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

While the above description contains many specific embodiments of the invention, these should not be construed as limitations on the scope of the invention, but rather as an example of one embodiment thereof. It is therefore to be understood that the present invention may be practiced otherwise than specifically described, without departing from the scope and spirit of the present invention. Thus, embodiments of the present invention should be considered in all respects as illustrative and not restrictive.

What is claimed is:

1. A non-compressive cervical collar for stabilizing a user's neck while maintaining a clearance zone along sides of the user's neck, the non-compressive cervical collar comprising:
a chest section configured to receive a user's chest, the chest section comprising a protrusion that is attached to at least one pillar;
a first shoulder section configured to receive a user's first shoulder and a second shoulder section configured to receive a user's second shoulder;
a jaw base that is attached to the at least one pillar, the jaw base comprising at least one jaw base attachment pad that attaches to at least one jaw strap, wherein the at least one jaw strap wraps around the user below a user's ears and above a user's shoulders without making contact with the sides of the user's neck; and
a back section comprising a top back section, the top back section comprising a top back attachment pad that attaches to the at least one jaw strap.

2. The non-compressive cervical collar of claim 1, wherein the at least one pillar comprises a first pillar and a second pillar.

3. The non-compressive cervical collar of claim 2, wherein the first and second pillars are separated by a distance creating an opening allowing access to a user's throat.

4. The non-compressive cervical collar of claim 1, wherein the at least one jaw base attachment pad comprises a first jaw base attachment pad and a second jaw base attachment pad, and wherein the at least one jaw strap comprises a first jaw strap and a second jaw strap.

5. The non-compressive cervical collar of claim 4, wherein the first jaw base attachment pad and the first jaw strap attach using a first jaw base hook-and-loop fastener system and the second jaw base attachment pad and the second jaw strap attach using a second jaw base hook-and-loop fastener system.

6. The non-compressive cervical collar of claim 5, wherein the top back section comprises at least one top back slot.

7. The non-compressive cervical collar of claim 6 further comprising a head strap that wraps around the user's head, wherein the at least one top back slot receives the head strap and the head strap folds and attaches onto itself.

8. The non-compressive cervical collar of claim 7, wherein the head strap attaches onto itself using a hook-and-loop fastener system.

9. The non-compressive cervical collar of claim 1, wherein the back section comprises a bottom back section, wherein the top back section receives a head of the user and the bottom back section receives a back of the user.

10. The non-compressive cervical collar of claim 1, wherein the first shoulder section comprises a first front shoulder section and a first back shoulder section.

11. The non-compressive cervical collar of claim 10, wherein the first front shoulder section and the first back shoulder section are connected.

12. The non-compressive cervical collar of claim 11, wherein the first front and back shoulder sections are connected using a first shoulder strap.

13. The non-compressive cervical collar of claim 11, wherein the second shoulder section comprises a second front shoulder section and a second back shoulder section.

14. The non-compressive cervical collar of claim 13, wherein the second front shoulder section and the second back shoulder section are connected.

15. The non-compressive cervical collar of claim 14, wherein the second front and back shoulder sections are connected using a second shoulder strap.

16. The non-compressive cervical collar of claim 1, wherein the jaw base further comprises a chin dip for receiving a user's chin.

17. The non-compressive cervical collar of claim 1, wherein the chin dip includes a cavity allowing a mouth of the user to open and close.

18. The non-compressive cervical collar of claim 1, wherein the chest section, the first and second shoulder sections, the jaw base, and the back section include padding for user comfort.

19. The non-compressive cervical collar of claim 1, wherein the chest section, first front and back shoulder sections, the second front and back shoulder sections, the jaw base, the first and second pillars, the top back section, the bottom back section are modular components.

20. The non-compressive cervical collar of claim 19, wherein the chest section comprises a first and second chest section that are modular components and mirror images of each other, the bottom back section comprises a first and second bottom back section that are modular components and mirror images of each other, the top back section comprises a first and second top back section that are modular components and mirror images of each other.
